(19) United States
(12) Patent Application Publication
(10) Pub. No.: US 2016/0213719 A1
(43) Pub. Date: Jul. 28, 2016

(54) ANTIVIRAL AGENT

(71) Applicants: DENSO CORPORATION, Aichi (JP); CHUBU UNIVERSITY EDUCATIONAL FOUNDATION, Aichi (JP)

(72) Inventors: Hiroki KURIYAMA, Kariya-city (JP); Kinya ATSUMI, Kariya-city (JP); Hiroaki FUKUDA, Kariya-city (JP); Kyoko HAYASHI, Imizu-shi (JP)

(21) Appl. No.: 15/023,780

(22) PCT Filed: Aug. 21, 2014
(86) PCT No.: PCT/JP2014/004305

§ 371 Date: Mar. 22, 2016

(30) Foreign Application Priority Data
Sep. 24, 2013 (JP) 2013-197102

(51) Int. Cl. A61K 36/05

(52) U.S. Cl. CPC A61K 36/05; A61K 2236/33; A61K 2236/331

(57) ABSTRACT

An antiviral agent according to the present disclosure includes, as an active ingredient, an extract extracted from a microalga Pseudochoricystis ellipsoidea Sekiguchi et Kurano gen. et sp. nov. MBIC11204 strain. The microalga may be cultured in a culture medium comprising sufficient nitrogen or may be cultured in a nitrogen-deficient medium thereafter. The extract is extracted from the microalga with alcohol, hot water, or the like as an extraction solvent. The extract may be extracted from the residue thereof with hot water. The antiviral agent may include the extract obtained using one extraction solvent, or may include a mixture of the extracts obtained using multiple extraction solvents.

### CROSS-REFERENCE TO RELATED APPLICATION

This application is based on Japanese Patent Application No. 2013-197102 filed on Sep. 24, 2013, the disclosure of which is incorporated herein by reference.

### TECHNICAL FIELD

The present disclosure relates to an antiviral agent.

### BACKGROUND ART

Conventionally, algae have been utilized in foods, feeds, or the like. As one method of utilizing algae, an antiviral agent including an extract from Coccomyxa alga as an active ingredient is disclosed (referring to Patent literature 1).

The inventors of the present application have found the following. Alga as disclosed in Patent literature 1 only has an antiviral activity against cells alone, and an alga that exhibits an antiviral activity in an in vivo test in consideration of various infection protective functions is not known.

### PRIOR ART DOCUMENT

### Patent Document

Patent literature 1: Japanese Patent No. 4411523 B

### SUMMARY OF INVENTION

It is an object of the present disclosure to provide a novel antiviral agent.

An antiviral agent according to one embodiment of the present disclosure includes, as an active ingredient, an extract from a microalga Pseudochoricystis ellipsoidea Sekiguchi et Kurano gen. et sp. nov. MBIC 11204 strain.

According to the antiviral agent of the present disclosure, it may be possible to obtain an antiviral activity in an in vivo test in consideration of various infection protective functions. A novel antiviral agent is provided.

### BRIEF DESCRIPTION OF DRAWINGS

The above and other objects, features and advantages of the present disclosure will become more apparent from the following detailed description made with reference to the accompanying drawings. In the drawings:

FIG. 1 is a graph showing results of recording severity (a lesion score) of genital herpes;

FIG. 2 is a graph showing the amount of a herpes simplex virus type 2 (HSV-2) in the genital organ on three days after infection;

FIG. 3 is a graph showing the amount of an influenza A virus (A/NWS/33 strain, subtype H1N1) on three days after infection;

FIG. 4 is a graph showing transition in a body weight of mice after infection with the influenza A virus;

FIG. 5 is a graph showing the neutralizing antibody titer in a mouse serum on 14 days after infection with the influenza A virus;

FIG. 6 is a graph showing the neutralizing antibody titer in the mouse bronchial lavage fluid (BALF) on 14 days after infection with the influenza A virus;

FIG. 7 is a graph showing the amount of an influenza A virus (A/NWS/33 strain, subtype H1N1) on three days after infection;

FIG. 8 is a graph showing the transition in the body weight of mice after infection with the influenza A virus;

FIG. 9 is a graph showing the neutralizing antibody titer in the mouse serum on 14 days after infection with the influenza A virus;

FIG. 10 is a graph showing the neutralizing antibody titer in the mouse bronchial lavage fluid (BALF) on 14 days after infection with the influenza A virus;

FIG. 11 is a graph showing the amount of IgA in the fecal extract on 14 days after infection with the influenza A virus;

FIG. 12 is a graph showing the amount of IgA in the mouse bronchial lavage fluid (BALF) on 14 days after infection with the influenza A virus;

FIG. 13 is a graph showing the transition in the body weight of mice after infection with the influenza A virus;

FIG. 14 is a graph showing the amount of an influenza A virus (A/NWS/33 strain, subtype H1N1) on three days after infection;

FIG. 15 is a graph showing the neutralizing antibody titer in the mouse serum on 14 days after infection with the influenza A virus;

FIG. 16 is a graph showing the neutralizing antibody titer in the mouse bronchial lavage fluid (BALF) on 14 days after infection with the influenza A virus;

FIG. 17 is a graph showing the amount of IgA in the mouse fecal extract on 14 days after infection with the influenza A virus; and

FIG. 18 is a graph showing the amount of IgA in the mouse bronchial lavage fluid (BALF) on 14 days after infection with the influenza A virus.

### PREFERRED EMBODIMENTS FOR CARRYING OUT INVENTION

Embodiments of the present disclosure will be described. The microalga Pseudochoricystis ellipsoidea Sekiguchi et Kurano gen. et sp. nov. MBIC 11204 strain that is used in the present disclosure was deposited in International Patent Organism Depositary (IPOD), National Institute of Advanced Industrial Science and Technology (Chuo 6, 1-1-1 Higashi, Tsukuba-shi, Ibaraki-ken, Japan) on Feb. 15, 2005 under the Accession Number FERM P-20401, and was transferred to the international deposit on Jan. 18, 2006 under the Accession Number FERM BP-10484 pursuant to the regulations under the Budapest Treaty.

The MBIC 11204 strain may be cultured in a culture medium including sufficient nitrogen, or may be cultured in a nitrogen-deficient medium thereafter. An extract (also referred to as “microalgae extract”) may be obtained by, for example, extraction from the MBIC 11204 strain with an alcohol (for example, ethanol), hot water, or the like as an extraction solvent. The extraction may be, for example, performed sequentially by using multiple types of extraction solvent. For example, extraction from the MBIC 11204 strain may be performed using an alcohol initially, and from the residue, extraction may be performed using hot water. The antiviral agent may include, for example, the extract obtained using one extraction solvent, or may include a mixture of each of the extracts obtained using multiple extraction solvents.

When extraction is performed, for example, the MBIC 11204 strain may be dried (for example, lyophilized), thereby preparing dried algal cells, and the extraction may be performed from the dried algal cells.

The extract may be, for example, fractionated by a method such as column chromatography. The antiviral agent may include the whole extract or may include partial fractions (for example, a fraction having a particularly high antiviral activity).

The dosage form of the antiviral agent is not particularly limited, and may be, for example, a liquid, a powder, a solid, or the like. The concentration of the extract in the antiviral agent is not particularly limited and may be appropriately determined according to volume, application, usage, or the like. The antiviral agent may include any of various components other than the extract as needed.

### EXAMPLES

(1) Cultivation of Microalga in Culture Medium Including Sufficient Nitrogen

500 mL of a culture medium having a composition shown in the following Table 1 was prepared using desalted water, and placed in a flat glass flask (working volume: 500 mL), and autoclaved.

TABLE 1
Composition of Culture Medium

| | | |
|---|---|---|
| NaNO₃ | 150 | mg |
| MgSO₄•7H₂O | 10 | mg |
| KH₂PO₄ | 3.5 | mg |
| K₂HPO₄ | 4.5 | mg |
| CaCl₂•2H₂O | 0.9 | mg |
| Fe-EDTA | 1.2 | mL |
| Metal solution (*) | 0.1 | mL |
| Desalted water | 99.8 | mL |

| | |
|---|---|
| pH | 7.5 |

(*) Metal solution

| | |
|---|---|
| H₃BO₃ | 7 mg |
| MgSO₄•7H₂O | 15 mg |
| ZnSO₄•7H₂O | 30 mg |
| CuSO₄•5H₂O | 30 mg |
| Na₂MoO₄ | 0.3 mg |
| CoCl₂ | 7 mg |
| Desalted water | 100 mL |

The MBIC 11204 strain was inoculated into the culture medium, and the flask was closed with an air-permeable stopper. Then, air supplemented with 3% CO₂ was introduced into the flask and at the same time, the culture solution in the flask was stirred. At this time, the flask was irradiated with light by a white fluorescent lamp from the outside of the flask. Furthermore, the temperature inside the flask was adjusted to around 28° C. (Celsius degree) by immersing the flask in a constant temperature water bath.

The dry weight of the algal cells was measured over time as an index of the growth of the MBIC 11204 strain. The specific growth rate at the logarithmic growth phase was 0.079 h⁻¹ and cell division occurred every 8.8 hours. After the algal cells were sufficiently grown, a 300 mL portion of the culture solution was collected. The algal cells were separated by centrifugation from the 300 mL portion of the culture solution. Thereafter, the algal cells were lyophilized, and 520.4 mg of dried algal cells S1 were obtained.

(2) Cultivation of Microalga in Nitrogen-Deficient Medium

The MBIC 11204 strain was cultured in 500 mL of a culture medium having a composition shown in the Table 1 in the same manner as in the above (1). A 400 mL portion of the culture solution was collected, and the algal cells were separated by centrifugation from the 400 mL portion of the culture solution. A condition for the centrifugation was set to 15,000 rpm for 10 minutes.

Subsequently, the centrifuged algal cells were washed twice with a nitrogen-deficient medium having a composition excluding NaNO₃ from the composition shown in the Table 1, and then further cultured for three days under the same conditions using the nitrogen-deficient medium. By doing this, a culture solution including algal cells with intracellular accumulation of a hydrocarbon was obtained. A 300 mL portion of the culture solution was collected, and the algal cells were separated by centrifugation from the collected 300 mL portion of the culture solution. Thereafter, the algal cells were lyophilized, and 884.7 mg of dried algal cells S2 were obtained.

(3) Extraction With Ethanol

1 L of ethanol was added to 100 g of the dried algal cells S1 to disperse the algal cells. The resulting dispersion was left to stand in a dark place for three days. The dispersion after being left to stand was filtered and separated into a primary filtrate and a residue. 1 L of ethanol was added to this residue in the same manner as described above to disperse the residue, and the resulting dispersion was left to stand for three days. Thereafter, the dispersion was filtered again and separated into a secondary filtrate and a residue. This filtration procedure was repeated once more, and a tertiary filtrate and a residue were obtained.

The primary filtrate, the secondary filtrate, and the tertiary filtrate were mixed, and ethanol was distilled off by an evaporator. The resulting residue was dried under reduced pressure, and 8.5 g of an ethanol extract DE was obtained. In addition, 8.7 g of an ethanol extract DE′ was obtained in the same manner as the method described above using the same amount of the dried algal cells S2 instead of the dried algal cells S1.

After 8.5 g of the ethanol extract DE was suspended in methanol, the resulting suspension was applied to a column packed with a synthetic adsorbent (DIAION HP-20, manufactured by Mitsubishi Chemical Corporation). The amount of the synthetic adsorbent was 500 g, the inner diameter of the column was 3.5 mm, and the axial length of the column was 60 cm.

The ethanol extract DE was fractionated by allowing a first developing solvent (distilled water), a second developing solvent (a solvent including methanol and distilled water at a volume ratio of 1:1), a third developing solvent (methanol), and a fourth developing solvent (acetone) in a volume of 1.5 L each to pass through the column in this order. Each fraction was dried under reduced pressure at room temperature, and fractionated ethanol extracts were obtained.

A component included in the fraction obtained with the first developing solvent was determined as an ethanol extract DE1. The amount of the obtained ethanol extract DE1 was 777.9 mg. A component included in the fraction obtained with the second developing solvent was determined as an ethanol extract DE2. The amount of the obtained ethanol extract DE2 was 139.1 mg. A component included in the fraction obtained with the third developing solvent was determined as an ethanol extract DE3. The amount of the obtained ethanol extract DE3 was 1.78 g. A component included in the fraction obtained with the fourth developing solvent was determined as an ethanol extract DE4. The amount of the obtained ethanol extract DE4 was 3.26 g.

2.1 g of the ethanol extract DE4 was applied to a column packed with a fractionation chromatographic silica gel (silica gel 60, manufactured by Merck Ltd.). The packing amount of the silica gel was 30 g, the particle size of the silica gel was from 0.063 to 0.200 mm, the inner diameter of the column was 2 cm, and the axial length of the column was 20 cm.

The ethanol extract DE4 was further fractionated by allowing developing solvents A to G to pass through the column in this order. The developing solvents A to G are the following solvents.

Developing solvent A: n-hexane

Developing solvent B: a solvent obtained by mixing n-hexane and ethyl acetate at a volume ratio of 5:1

Developing solvent C: a solvent obtained by mixing n-hexane and ethyl acetate at a volume ratio of 3:1

Developing solvent D: a solvent obtained by mixing n-hexane and ethyl acetate at a volume ratio of 1:1

Developing solvent E: a solvent obtained by mixing n-hexane and ethyl acetate at a volume ratio of 1:3

Developing solvent F: ethyl acetate

Developing solvent G: a solvent obtained by mixing ethyl acetate and methanol at a volume ratio of 1:1

The fraction obtained with the developing solvent A was dried under reduced pressure at room temperature, and 216.3 mg of an extract (hereinafter, referred to as “ethanol extract DE4-a”) was obtained. Further, the fraction obtained with the developing solvent E was dried under reduced pressure at room temperature, and 170.3 mg of an extract (hereinafter, referred to as “ethanol extract DE4-b”) was obtained.

(4) Extraction With Hot Water

1 L of distilled water was added to the residue after extracting the ethanol extract DE from the dried algal cells S1 in the above (3), and the resulting dispersion was heated at 85° C. for 1 hour and then filtered, and the dispersion was separated into a primary filtrate and a residue. Then, 1 L of distilled water was added to the residue to disperse the residue, and the resulting dispersion was heated again at 85° C. for 1 hour and then filtered, and the dispersion was separated into a secondary filtrate and a residue.

The obtained primary filtrate and the secondary filtrate were mixed, and the resulting mixture was concentrated under reduced pressure at room temperature, followed by lyophilization. 17.0 g of a hot-water extract DW was obtained.

Subsequently, 4 L of ethanol was added to the hot-water extract DW, and the mixture was left to stand overnight at 4° C. The mixture after being left to stand was separated into a supernatant and a precipitate. The supernatant was concentrated under reduced pressure at room temperature, followed by lyophilization, and 7.5 g of a supernatant component DL was obtained. The precipitate was separated by centrifugation, and the obtained precipitate was washed with 1 L of ethanol and thereafter dissolved in 1 L of distilled water, followed by lyophilization, and 8.2 g of a precipitate component DH was obtained.

Further, 1 L of distilled water was added to the residue after extracting the ethanol extract DE′ from the dried algal cells S2 in the above (3). The resulting dispersion was heated at 85° C. for 1 hour and then filtered, and the dispersion was separated into a primary filtrate and a residue. Then, 1 L of distilled water was added to the residue to disperse the residue, and the resulting dispersion was heated again at 85° C. for 1 hour and then filtered, and the dispersion was separated into a secondary filtrate and a residue. The obtained primary filtrate and the secondary filtrate were mixed, and the resulting mixture was concentrated under reduced pressure at room temperature, followed by lyophilization, and 10.5 g of a hot-water extract DW′ was obtained.

(5) Extraction With Hot Water From Oil Extraction Residue

500 mL of n-hexane was added to 19.7 g of the dried algal cells S2 to disperse the dried algal cells S2. Then, the resulting dispersion was left to stand at room temperature for 1 day, and an oil extraction was performed. The dispersion after being left to stand was filtered and separated into a filtrate including an oil and an oil extraction residue. 500 mL of n-hexane was added to this residue to disperse the residue, and the resulting dispersion was left to stand for 1 day. Thereafter, the dispersion was filtered again and separated into a filtrate and an oil extraction residue. This procedure was repeated once more, and a filtrate and an oil extraction residue were obtained.

The oil extraction residue was dried under reduced pressure at room temperature. 1 L of distilled water was added to the obtained oil extraction residue, and the dispersion was heated at 85° C. for 1 hour and then filtered. The dispersion was separated into a primary filtrate and a residue. Then, 1 L of distilled water was added to the residue to disperse the residue in the same manner as described above, and the resulting dispersion was heated again at 85° C. for 1 hour and then filtered. The dispersion was separated into a secondary filtrate and a residue. The thus obtained primary filtrate and the secondary filtrate were mixed, and the resulting mixture was concentrated under reduced pressure at room temperature, followed by lyophilization, and 7.0 g of a hot-water extract DO from the oil extraction residue was obtained.

(6) Test for Confirming Effect of Antiviral Agent on Herpes Simplex Virus Type 2 (HSV-2)

The ethanol extracts DE, DE2, DE4, DE4-a, and DE4-b were used as samples, and the effect on HSV-2 was tested according to the following method.

HSV-2 was inoculated into BALB/c mice (female, at 5 weeks of age) (n=5) to infect the mice with HSV-2. HSV-2 was locally administered once to the mice. The single dose was set to 1×10⁴ PFU/20 μL/mouse. The dose of “1×10⁴ PFU/20 μL/mouse” means that a solution obtained by adding 1×10⁴ PFU of the virus to 20 μL of phosphate buffered saline (PBS) is administered to one mouse. The “PFU” stands for “plaque forming unit”.

Incidentally, medroxyprogesterone 17-acetate (3 mg/0.1 mL/mouse) was subcutaneously injected into each mouse on six days before and one day before virus inoculation.

Further, each of the samples was locally administered twice a day to each mouse from one hour before the virus inoculation to seven days after the virus inoculation. The single dose of the sample was set to 1 mg/20 μL/mouse. The dose of “1 mg/20 μL/mouse” means that a solution obtained by dissolving 1 mg of the sample in 20 μL of PBS supplemented with 1% DMSO is administered to one mouse.

The local area of each mouse was washed with PBS on three days after inoculation of the virus, and the amount of the virus therein was measured by a plaque method. A significant difference in the amount of the virus compared with the control group was represented as follows: *P<0.05, **“P<0.01, and ***P<0.001.

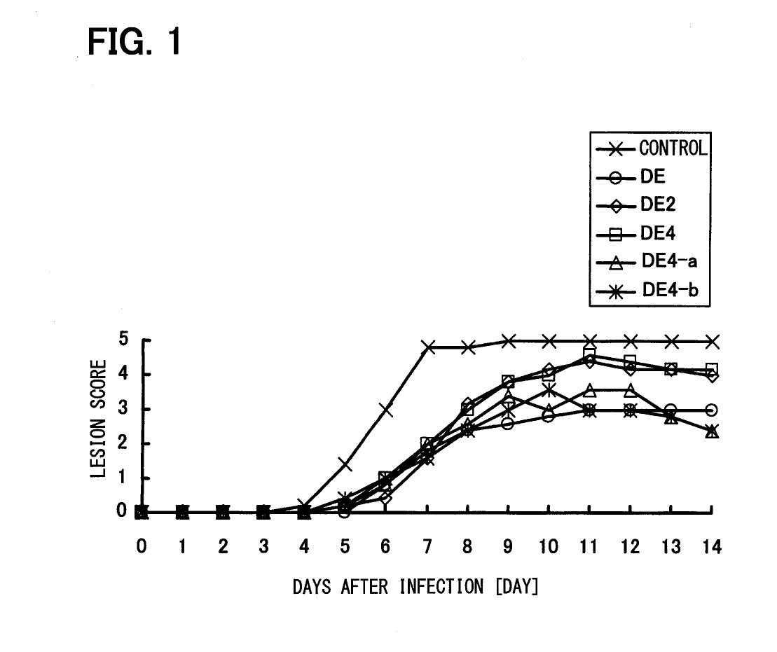

Further, fatal cases and the degree of pathogenesis (a lesion score) of genital herpes were recorded from the day of inoculation of the virus to 14 days thereafter. The results of recording the lesion score are shown in FIG. 1. The lesion scores “1” to “5” in FIG. 1 indicate the following meanings.

1: with swelling

2: with swelling and redness

3: with fluid exudation

4: hind leg paralysis

5: death

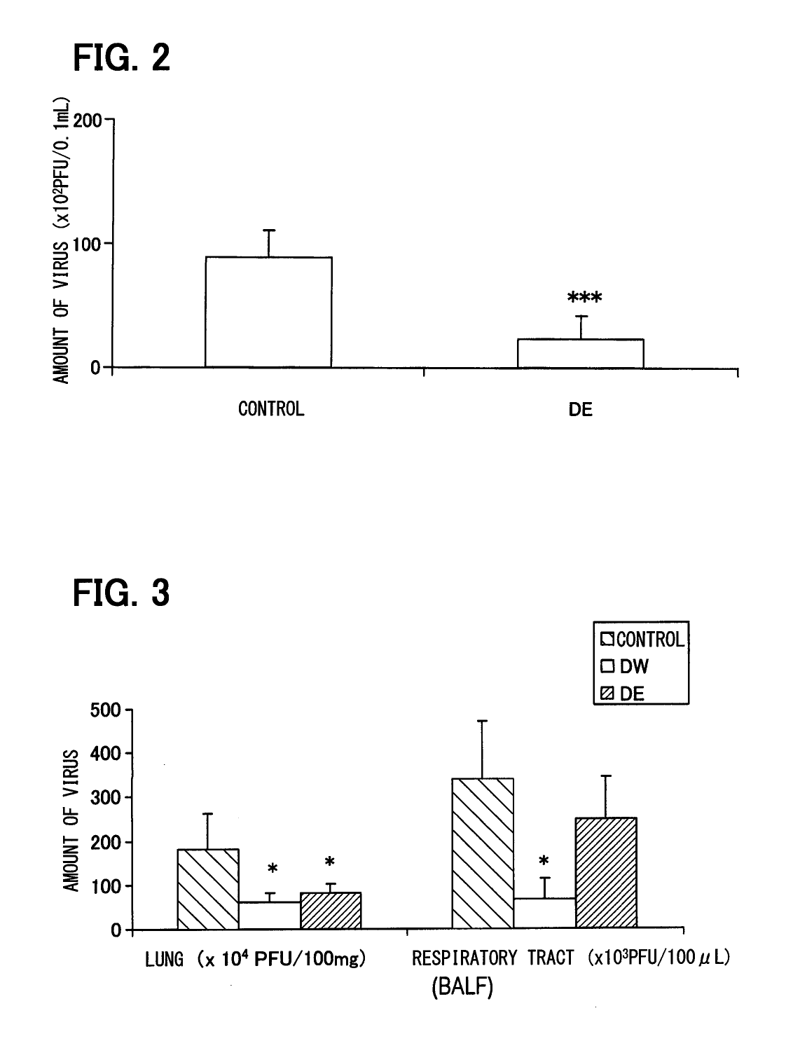

It was found from these results that the ethanol extracts DE, DE2, DE4, DE4-a, and DE-4b suppress the degree of occurrence of the herpes virus. Further, the amount of the virus on three days after infection is shown in FIG. 2. It was found from these results that the ethanol extract DE significantly decreases the production amount of the virus.

(7) Test for Confirming Effect of Antiviral Agent on Influenza A Virus

The ethanol extracts DE and DE′, the hot-water extracts DW, DW′, and DO, the supernatant component DL, and the precipitate component DH were used as samples, and the effect on an influenza A virus was tested according to the following method.

(7-1) Measurement of Body Weight, Amount of Virus, and Neutralizing Antibody Titer

An influenza A virus (A/NWS/33 strain, subtype H1N1) was inoculated into BALB/c mice (female, at 6 weeks of age) (n=10) through the nose to infect the mice with the influenza A virus. The inoculation amount of the virus was set to 1×10⁴ PFU/50 μL/mouse. The inoculation amount of “1×10⁴ PFU/50 μL/mouse” means that a solution obtained by adding 1×10⁴ PFU of the virus to 50 μL of PBS is administered to one mouse.

Each sample was orally administered to the mice twice a day (9 o'clock and 18 o'clock) for 2 weeks from 1 week before to 1 week after the day of inoculation of the virus. The dose of the sample was set to 5 mg/day.

The body weight of each mouse and the number of dead mice were recorded for 2 weeks from the day of inoculation of the virus. The fatal case was not observed in the groups in which each of the ethanol extract DE, the hot-water extract DW, the supernatant component DL, and the precipitate component DH was administered as the sample. One mouse died six days after infection in the control group.

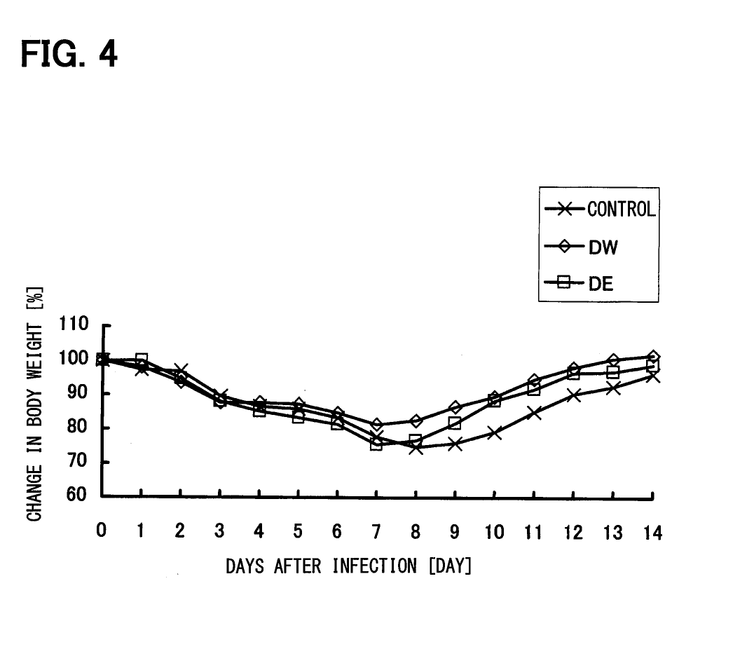

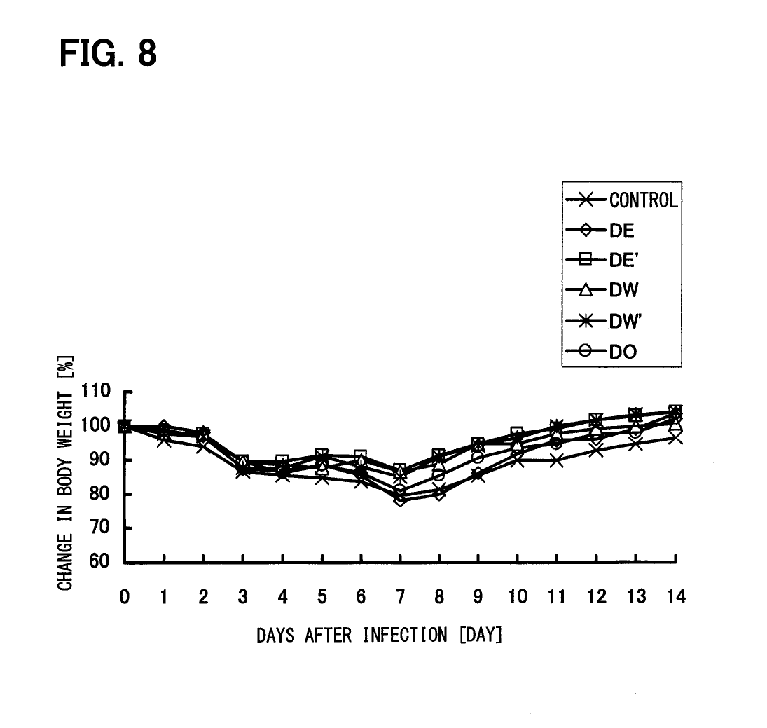

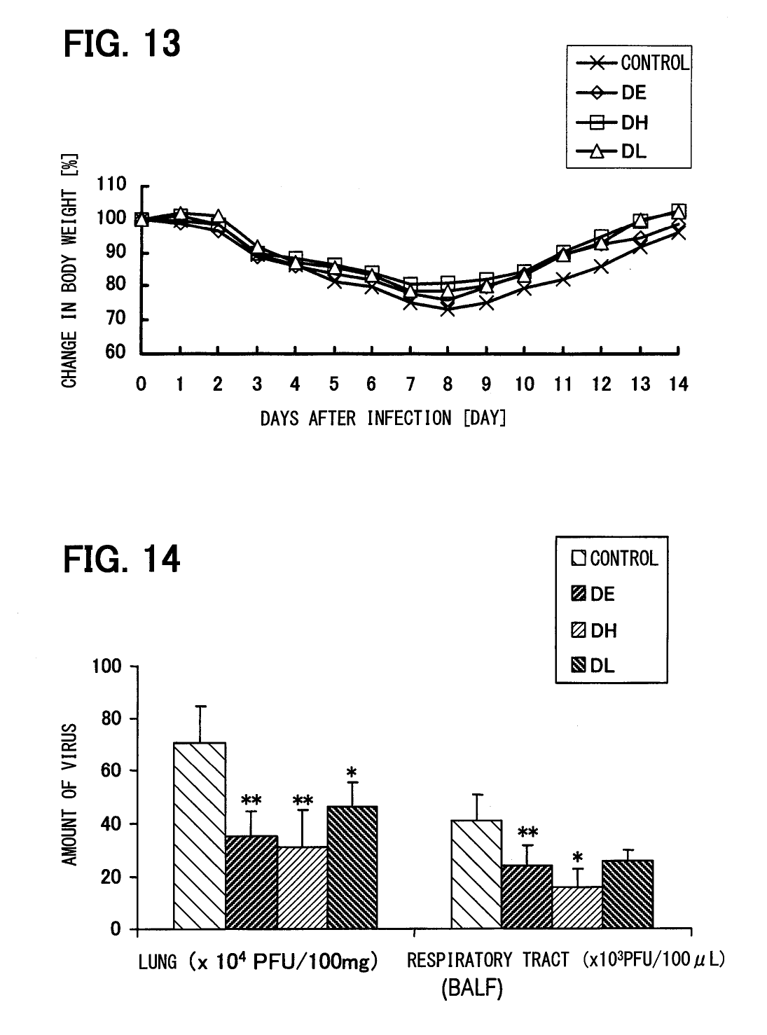

The transition in the body weight of mice is shown in FIG. 4, FIG. 8, and FIG. 13. As shown in FIG. 4, in the groups in which each of the ethanol extract DE and the hot-water extract DW was administered as the sample, a decrease in the body weight comparable to that in the control group was observed up to 7 days after infection, however, the recovery of the body weight from 8 days after infection was faster than in the control group. In particular, the recovery was much faster in the group in which the ethanol extract DE was administered.

As shown in FIG. 8, in the groups in which each of the ethanol extract DE and the hot-water extracts DW, DW′, and DO was administered as the sample, the decrease in the body weight up to 7 days after infection was less than in the control group. The recovery of the body weight thereafter was faster than in the control group. Further, in the group in which the ethanol extract DE′ was administered as the sample, the transition in the body weight up to 10 days after infection was comparable to that in the control group, however, the recovery thereafter was faster than in the control group.

As shown in FIG. 13, in the groups in which each of the ethanol extract DE, the supernatant component DL, and the precipitate component DH was administered as the sample, the decrease in the body weight was less than in the control group.

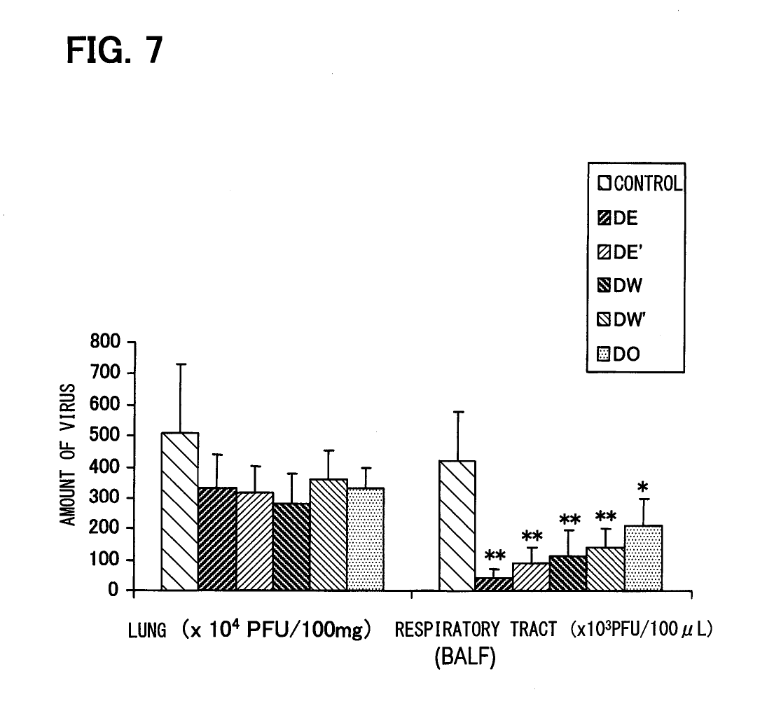

The mouse bronchial lavage fluid (BALF) and the lung were collected from each of the half mice (5 mice) on three days after the day of inoculation of the virus. The BALF is a fluid obtained by introducing 0.8 mL of ice-cooled PBS into the respiratory tract through a catheter for washing the respiratory tract. After collecting the BALF, the lung was excised. For each of the lung and the BALF, the amount of the virus included therein was measured. The amount of the virus on three days after infection is shown in FIG. 3, FIG. 7, and FIG. 14.

As shown in FIG. 3, in the groups in which each of the ethanol extract DE and the hot-water extract DW was administered as the sample, the growth of the virus was suppressed in comparison with the control group. In particular, the growth of the virus was remarkably suppressed in the group in which DW was administered.

As shown in FIG. 7, in the groups in which each of the ethanol extracts DE and DE′ and the hot-water extracts DW, DW′, and DO was administered as the sample, the growth of the virus was suppressed in comparison with the control group.

As shown in FIG. 14, in the groups in which each of the ethanol extract DE, the supernatant component DL, and the precipitate component DH was administered as the sample, the growth of the virus was suppressed in comparison with the control group.

From the rest of the half mice, the blood, the BALF, and the feces were collected 14 days after the day of inoculation of the virus. By using these blood and BALF, the neutralizing antibody titer was measured as shown below.

The serum was separated from the blood by centrifugation and inactivated. The conditions for the centrifugation at this time were set to 3,000 rpm at 4° C., and the conditions for the inactivation were set to 56° C. for 30 minutes. The BALF was stored at -80° C. immediately after collection.

A solution obtained by appropriately diluting the serum with PBS (hereinafter referred to as “diluted serum solution”) and a solution obtained by appropriately diluting the BALF with PBS (hereinafter referred to as “diluted BALF solution”) were prepared, respectively.

Then, 100 μL of the diluted serum solution and 100 μL of a virus solution (a solution including the influenza A virus at a concentration of 2,000 PFU/mL) were added to a 96-well plate and mixed with each other. This mixed solution is referred to as “serum-virus mixed solution”. The serum-virus mixed solution has a volume of 200 μL and includes 200 PFU of the influenza A virus.

Further, 100 μL of the diluted BALF solution and the 100 μL of the virus solution were added to a 96-well plate and mixed with each other.

This mixed solution is referred to as “BALF-virus mixed solution”. The BALF-virus mixed solution has a volume of 200 μL and includes 200 PFU of the influenza A virus.

As a control, 100 μL of PBS and 100 μL of the virus solution were added to a 96-well plate and mixed with each other. This mixed solution is referred to as “control solution”. The control solution has a volume of 200 μL and includes 200 PFU of the influenza A virus.

The serum-virus mixed solution, the BALF-virus mixed solution, and the control solution were treated at 37° C. for 1 hour. Then, MDCK cells cultured in a monolayer in 35-mm dishes were infected with the virus at room temperature by adding each of the solutions in an amount of 100 μL/dish.

After 1 hour, the MDCK cells were overlaid with an agar medium (2 mL/dish) and cultured at 37° C. for 2 days. After the medium was removed, the cells were fixed and stained with a crystal violet solution, and the number of plaques was counted. The number of plaques in each of the serum-virus mixed solution and the BALF-virus mixed solution was calculated. A 50% inhibitory dilution was calculated when the number of plaques in the control solution was taken as 100%, and determined as a neutralizing antibody titer.

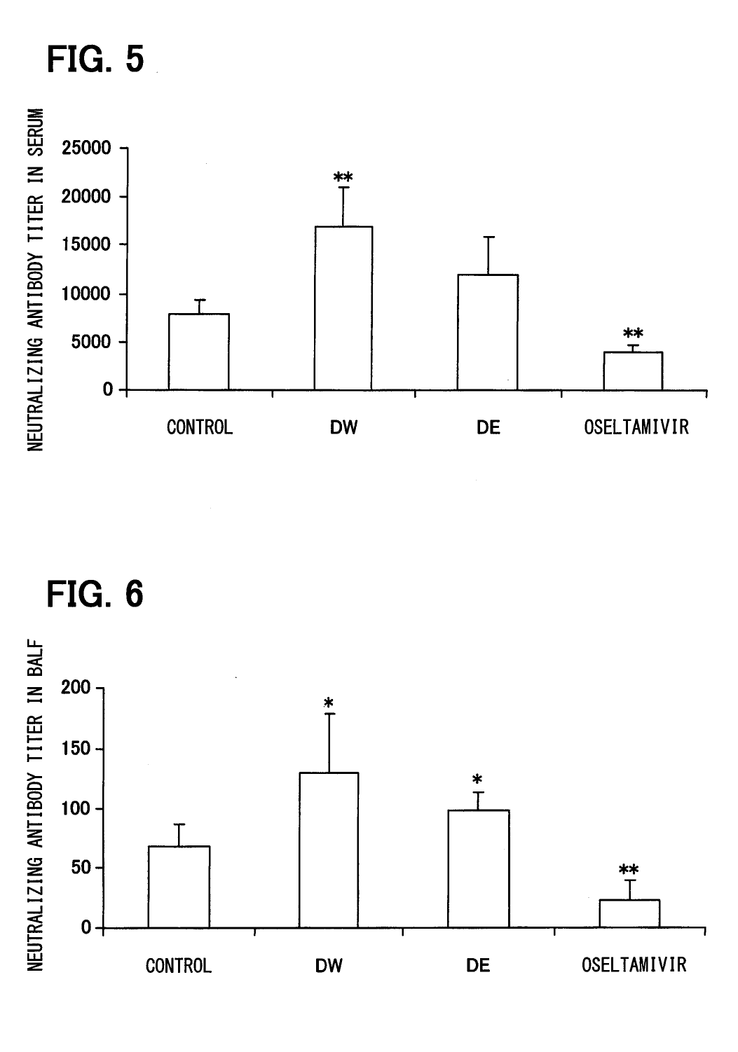

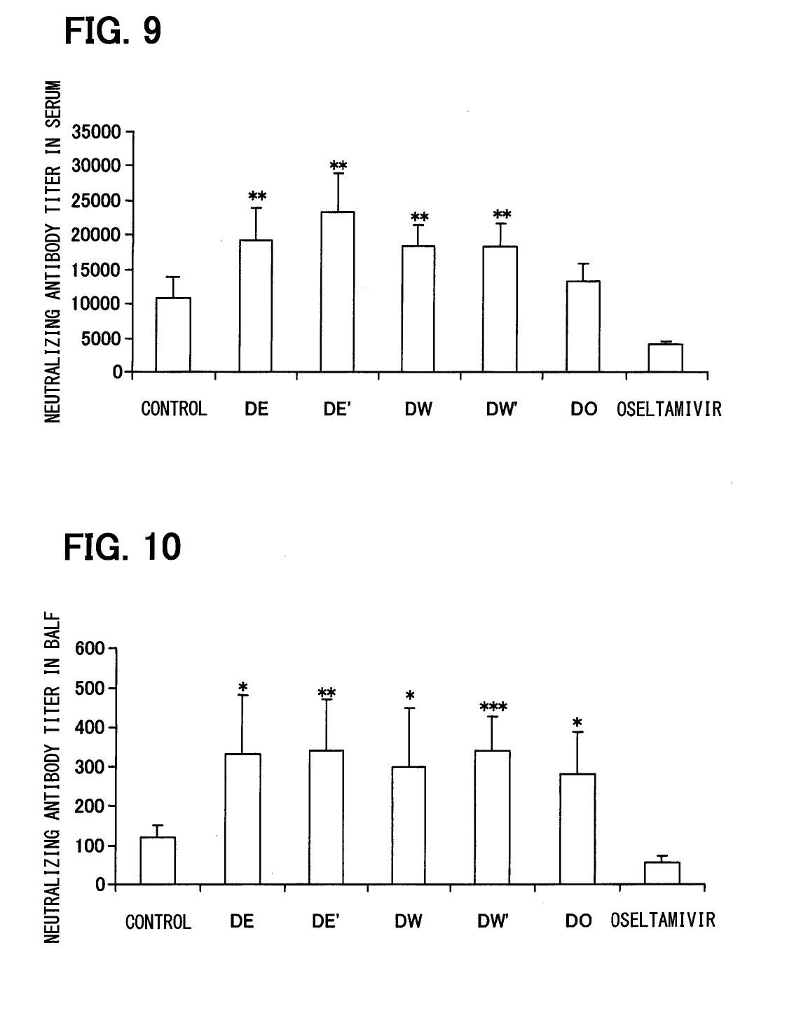

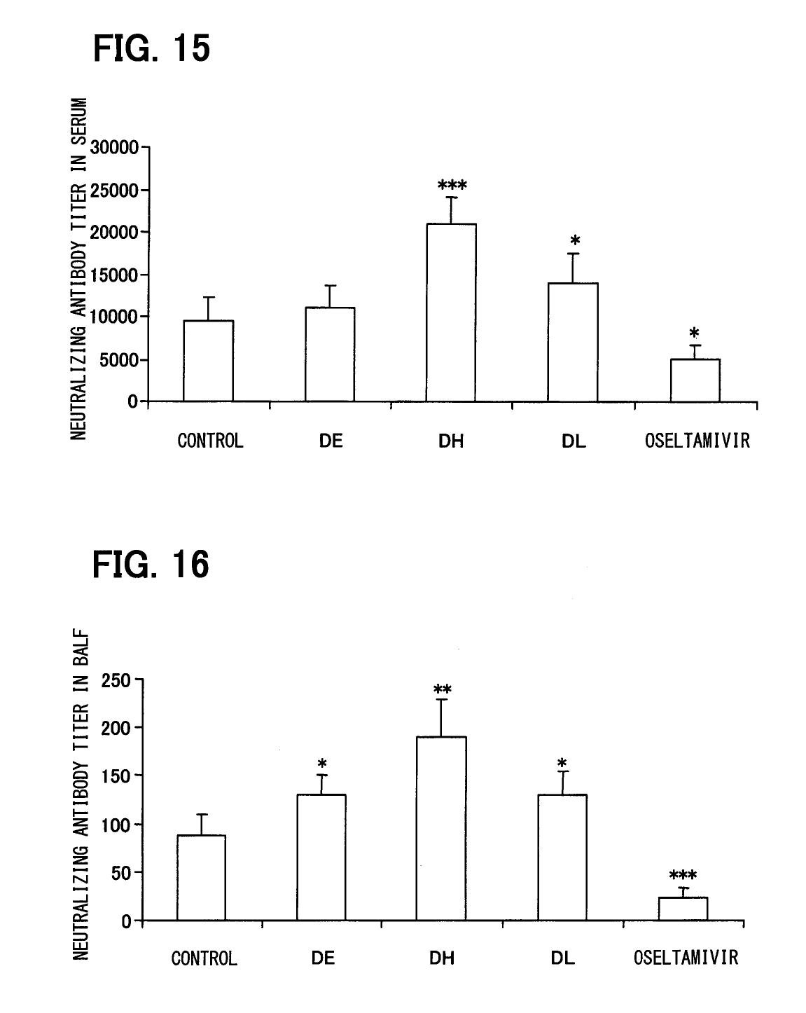

The neutralizing antibody titer in the serum-virus mixed solution on 14 days after infection is shown in FIG. 5, FIG. 9, and FIG. 15. In addition, the neutralizing antibody titer in the BALF-virus mixed solution on 14 days after infection is shown in FIG. 6, FIG. 10, and FIG. 16.

As shown in FIG. 5 and FIG. 6, in the groups in which each of the ethanol extract DE and the hot-water extract DW was administered as the sample, the neutralizing antibody titer was higher than in the control group and the group in which oseltamivir (manufactured by F. Hoffmann-La Roche, Ltd.) was administered.

As shown in FIG. 9 and FIG. 10, in the groups in which each of the ethanol extracts DE and DE′, and the hot-water extracts DW, DW′, and DO was administered as the sample, the neutralizing antibody titer was higher than in the control group and the group in which oseltamivir was administered.

As shown in FIG. 15 and FIG. 16, in the groups in which each of the ethanol extract DE, the supernatant component DL, and the precipitate component DH was administered as the sample, the neutralizing antibody titer was higher than in the control group and the group in which oseltamivir was administered.

Incidentally, a reason the neutralizing antibody titer is increased as described above is presumably that the ethanol extracts DE and DE′, the hot-water extracts DW, DW′, and DO, the supernatant component DL, and the precipitate component DH have an immune stimulatory effect.

(7-2) Measurement of IgA

To the feces collected 14 days after the day of inoculation of the virus, a 10-fold amount of PBS was added. The resulting mixture was left at room temperature for 15 minutes and then treated with a vortex mixer. The mixture was further left for 15 minutes and then centrifuged (3,000 rpm, 10 minutes), thereby obtaining a supernatant. This supernatant is referred to as “fecal extract”. The BALF collected 14 days after the day of inoculation of the virus was diluted to 5-fold with PBS. This diluted solution is referred to as “5-fold diluted BALF”.

Subsequently, ELISA was performed according to the following procedure, and IgA was measured.

a) An antigen (a purified virus at 1 μg/mL of PBS) was added to an ELISA 96-well plate (MaxiSorp, manufactured by Nunc, Inc.) at 50 μL/well and the plate was treated at 37° C. for 1 hour.

b) Each well was washed three times with a PBS-T solution. Here, the PBS-T solution is a PBS solution including polyoxyethylene (20) sorbitan monolaurate (product name: Tween-20) at a concentration of 0.5%.

c) 5% skim milk (dissolved in PBS) was added at 100 μL/well, and a blocking treatment was performed overnight at 4° C.

d) Each well was washed three times with PBS-T.

e) The fecal extract or the 5-fold diluted BALF was added at 50 μL/well and the plate was treated at 37° C. for 1 hour.

f) Each well was washed three times with PBS-T.

g) A secondary antibody (HRP-conjugated anti-mouse IgA, manufactured by Bethyl Laboratories, Inc.) was added at 50 μL/well and the plate was treated at 37° C. for 1 hour.

h) Each well was washed three times with PBS-T.

i) A substrate solution (0.4 mg/mL o-phenylenediamine+10 μL/mL of H₂O₂) was added at 50 μL/well and the plate was left at room temperature for 20 minutes.

j) The reaction was stopped by adding 4 N sulfuric acid at 25 μL/well.

k) An absorbance at 490 nm was measured. In the case of the fecal extract, the fecal extract was divided into three tubes, and the measurement was performed for each tube. In the case of the 5-fold diluted BALF, the measurement was performed twice for each sample. By using the average of the measurements, the amount of IgA was obtained from a calibration curve.

The calibration curve was created as follows. The mouse IgA (manufactured by Bethyl Laboratories, Inc.) was prepared at the following concentrations: 6.25, 12.5, 25, 50, 100, 200, 1000, and 5000 ng/mL. Then, mouse IgA at each concentration was added to an ELISA 96-well plate at 50 μL/well (3 wells per concentration), and the plate was treated at 37° C. for 1 hour. The procedures described in the above b), c), d), and g) to k) were performed. The calibration curve was created from the amount of IgA and the measurements of absorbance at 490 nm.

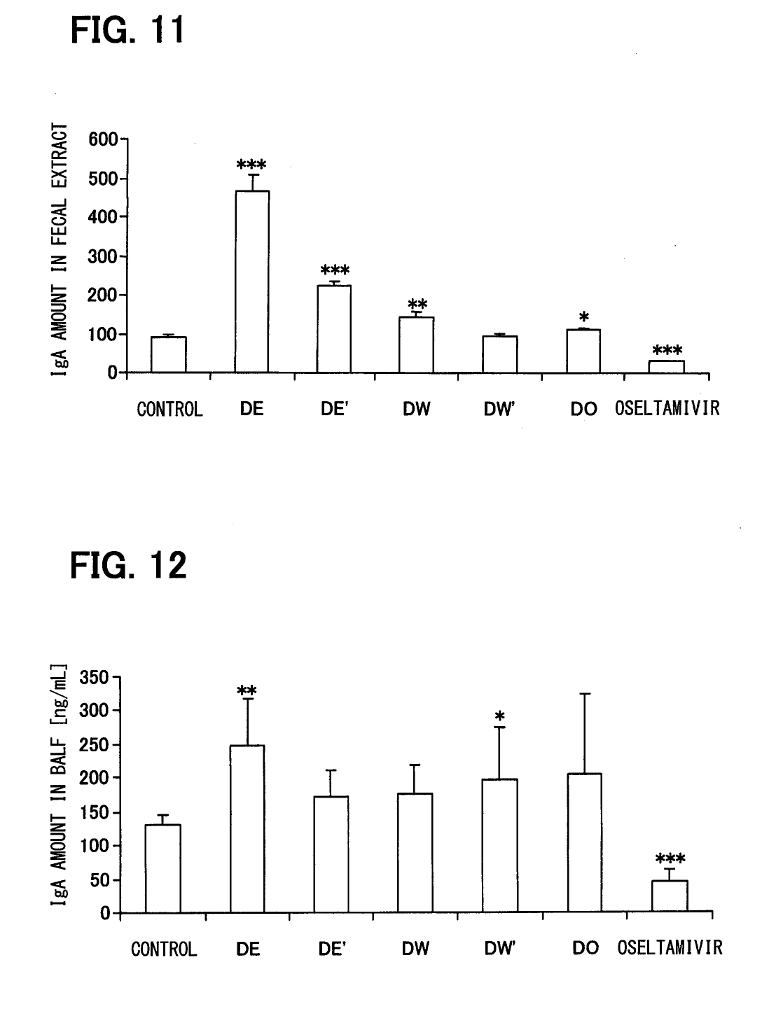

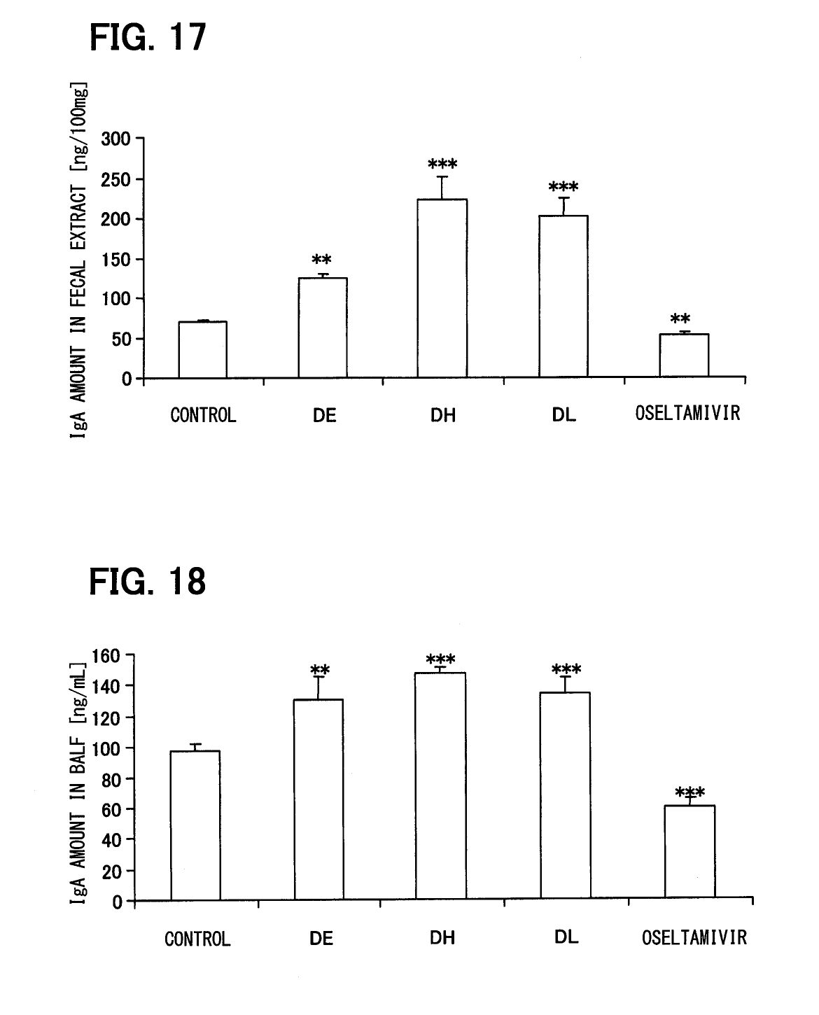

The amount of IgA in the fecal extract on 14 days after infection is shown in FIG. 11 and FIG. 17. Further, the amount of IgA in the 5-fold diluted BALF on 14 days after infection is shown in FIG. 12 and FIG. 18.

As shown in FIG. 11 and FIG. 12, in the groups in which each of the ethanol extracts DE and DE′, and the hot-water extracts DW, DW′, and DO was administered as the sample, the amount of IgA was higher than in the control group and the group in which oseltamivir was administered. In particular, the increase in the amount of IgA was remarkable in the group in which the ethanol extract DE was administered.

As shown in FIG. 17 and FIG. 18, in the groups in which each of the ethanol extract DE, the supernatant component DL, and the precipitate component DH was administered as the sample, the amount of IgA was higher than in the control group and the group in which oseltamivir was administered.

The antiviral agent according to the present disclosure includes an extract extracted from a microalga Pseudochoricystis ellipsoidea Sekiguchi et Kurano gen. et sp. nov. MBIC 11204 strain as an active ingredient. The antiviral agent according to the present disclosure has a high antiviral activity.

While the embodiments, the configurations, and the modes of the antiviral agent according to the present disclosure are illustrated above, embodiments, configurations, and modes according to the present disclosure are not limited to the respective embodiments, the respective configurations, and the respective modes described above. For example, an embodiment, a configuration, and an aspect which are obtained by appropriately combining technical portions disclosed in different embodiments, configurations, and aspects are also included in the embodiments, the configurations, and the aspects according to the present disclosure.

1. An antiviral agent comprising:
an extract from a microalga Pseudochoricystis ellipsoidea Sekiguchi et Kurano gen. et sp. nov. MBIC 11204 strain as an active ingredient.

2. The antiviral agent according to claim 1, wherein:
the extract from the microalga is provided by an extract that is extracted from the microalga with an alcohol.

3. The antiviral agent according to claim 2, wherein:
the alcohol is ethanol.

4. The antiviral agent according to claim 1, wherein:
the extract from the microalga is extracted with hot water from a microalga product after extraction from the microalga with an alcohol.

5. The antiviral agent according to claim 4, wherein:
the hot water is at 85° C.

6. The antiviral agent according to claim 1, wherein:
the antiviral agent is directed to a herpes simplex virus type 2 and an influenza A virus.
